(19) United States
(12) Patent Application Publication
(10) Pub. No.: US 2026/0066074 A1
(43) Pub. Date: Mar. 5, 2026

(54) METHODS AND SYSTEMS FOR QUALITY CONTROL REVIEW OF ECG ANALYSES

(71) Applicant: KONINKLIJKE PHILIPS N.V., EINDHOVEN (NL)

(72) Inventors: Man Ching Cheung, Cambridge, MA (US); Jonathan Kamerman, Andover, MA (US)

(21) Appl. No.: 19/318,609

(22) Filed: Sep. 4, 2025

Related U.S. Application Data
(60) Provisional application No. 63/690,454, filed on Sep. 4, 2024.

(51) Int. Cl. G16H 15/00; G16H 50/70; G16H 80/00

(52) U.S. Cl. CPC G16H 15/00; G16H 50/70; G16H 80/00

(57) ABSTRACT

A method for reviewing ECG studies, comprising: selecting one or more ECG studies from a database of ECG studies, wherein the one or more ECG studies have been subjected to a first review by a first clinician; storing the selected one or more ECG studies in a review database; presenting the one or more selected ECG studies to a second clinician for a second review; storing the second review in the review database; comparing the first review and the second review for each of the one or more selected ECG studies to: (i) identify one or more differences between the first review and the second review, and/or (ii) identify a degree of difference of each of the identified one or more differences between the first review and the second review; generating a report summarizing the differences and/or degree of differences for the one or more selected ECG studies.

### FIELD OF THE DISCLOSURE

The present disclosure is directed generally to methods and systems for improved quality control review of ECG analyses.

### BACKGROUND

The current market for Diagnostic Study Management software (e.g., ECG management) consists of products that route the signals containing diagnostic information (also called studies) generated from medical devices to physicians for diagnostic interpretation using tools built specifically for that diagnostic interpretation. It is common, especially in the ECG market, for machine generated measurements and algorithms to generate preliminary diagnostic clinical statements.

Typically, the clinical decision support provided by these algorithms requires medical device clearance by government entities before they are used for clinical diagnostic purposes. The process of clearing new versions of algorithms is costly, lengthy, and clinically important. The primary reason clearance is costly is that each new algorithm needs to be reviewed by multiple experts in the field of medicine related to the algorithm. This mirrors quality processes in the healthcare system, where, from a continuing education, improvement, and safety perspective, it is of benefit for review and discussion to occur. For example, diagnostic studies can be set aside where a second expert corrects the original review, and a moderator adjudicates a final decision when the evaluation of the first and second experts are not fully aligned.

If any part of the signal needs to be modified to improve the adjudicated outcome, the process of evaluation needs to restart. This process is heavily documented, often manually, upon completion of the diagnostic review, the documentation is submitted for review by the government agency. A similar process-typically referred to as peer review—is often used by healthcare entities in diagnostic quality improvement and training programs. This is a voluntary process where physician overreading and adjudication is used to help physicians and specialists improve the diagnostic accuracy of reports. However, peer review is a highly manual process of comparing different versions of a diagnostic report as it progresses through the reading cycle. Every part of this process is cumbersome.

Peer review is often utilized in the ECG space. Some platforms for ECG diagnosis contain metadata regarding whether algorithmically-generated diagnostic ECG statements were accepted/modified/rejected by clinical users. However, it is unknown if the readers themselves were performing at a higher accuracy than the algorithms, or whether the resulting changes were clinical significant or meaningful. For example, the former can happen if fellows and residents (i.e. trainees) were assigned ECGs. These platforms do not track the types of corrections made by an attending physician, rather they only provide a final read and how it differed from algorithmic review.

Most diagnostic platforms do provide a mechanism for an attending physician to overread (or to perform a second read, or to make amendments) signed reports. This is a part of the overall diagnostic workflow and is an important built in quality check that depends on self-modulation by physician groups at hospitals. However, what is needed is a specific solution where large numbers of diagnostic data can be reviewed and that is separate from regular clinical workflow. The studies need to be flagged or identified in such a way that these “educational” reads do not impair care delivery (e.g., not interrupt urgent care situations) and is clearly marked so that the physicians attend to them when time is available. Subsequent to the peer review, there should also be a way for the quality director to review results without having to look through and re-assess all the peer reviewed studies. The problem is not as simple as identifying the differences. Diagnostic descriptions can overlap in semantic meaning but not in words (for example, atrial fibrillation and multifocal atrial tachycardia identify similar pathologies). In addition, the platforms themselves provide the ability for clinicians to add free-text descriptions, resulting in differences with varying degrees of clinical significance.

Further, the information from review is not stored in a way that allows for ease of reconstructing where the over-reader differed from their colleague in interpreting the ECG. At least one current approach requires significant data engineering and clinical expertise to assess audit logs to determine the sequence of modifications, stored within supporting data tables. Upon the provisional completion of a quality data table, the engineer must then work with the physician to ensure that the reverse-engineered sequence matches the actions taken by the over-reader. This process imposes a significant burden on the physician and increases the cost to deliver, as this process must be performed for each customer, with an employee who has expertise in both data and clinical domains.

### SUMMARY OF THE DISCLOSURE

Accordingly, there is a continued need for methods and systems for more efficient and less burdensome quality control review of ECG analyses, distinct from regular “reading” of diagnostic studies in the course of health care delivery.

Various embodiments and implementations herein are directed to methods and systems for reviewing ECG studies. An ECG review system selects one or more ECG studies from a database of ECG studies, wherein the one or more ECG studies have been subjected to a first review by a first clinician, and stores the selected ECG studies in a review database. The system then presents the one or more selected ECG studies to a second clinician for a second review, and stores the second review in the review database. A third entity compares the first review and the second review for each of the one or more selected ECG studies to: (i) identify one or more differences between the first review and the second review, and (ii) identify a degree of difference of each of the identified one or more differences between the first review and the second review. The system then generates a report summarizing the differences and degree of differences for the one or more selected ECG studies.

Generally, in one aspect, a method for reviewing ECG studies is provided. The method includes: (i) selecting one or more ECG studies from a database of ECG studies, wherein the one or more ECG studies have been subjected to a first review by a first clinician, the first review comprising one or more first review elements, the one or more first review elements comprising one or more of an identity of the first clinician, a diagnosis, annotation of the ECG study, a note associated with the ECG study, and a measurement associated with the ECG study; (ii) storing the selected one or more ECG studies, including the first review, in a review database; (iii) presenting the one or more selected ECG studies to a second clinician for a second review, the second review comprising one or more second review elements, the one or more second review elements comprising one or more of an identity of the second clinician, a diagnosis, annotation of the ECG study, a note associated with the ECG study, and a measurement associated with the ECG study; (iv) storing the second review in the review database; (v) comparing, by a third entity different from the first clinician and second clinician, the first review and the second review for each of the one or more selected ECG studies to: (1) identify one or more differences between the first review and the second review, and/or (2) identify a degree of difference of each of the identified one or more differences between the first review and the second review; and (vi) generating a report summarizing the differences and/or degree of differences for the one or more selected ECG studies.

According to an embodiment, the method further includes processing the first review and second review to align the one or more first review elements and the one or more second review elements.

According to an embodiment, the method further includes comparing, by a trained comparison algorithm, the first review and the second review for each of the one or more selected ECG studies to rank the one or more selected ECG studies, wherein the rank is utilized to: (i) present the one or more selected ECG studies in a ranked order for comparison, and/or (ii) eliminate any of the one or more selected ECG studies that fall below a predetermined ranking.

According to an embodiment, the third entity is a third clinician, and the comparing is performed manually by the third clinician using an adjudication viewer.

According to an embodiment, the third entity is a trained comparison algorithm.

According to an embodiment, the report comprises a performance metric based on the summarized differences and degree of differences for the one or more selected ECG studies.

According to an embodiment, the one or more ECG studies are randomly selected from the database of ECG studies.

According to an embodiment, the one or more ECG studies are selected from the database of ECG studies based on one or more criteria, the one or more criteria determined via a user interface prior to selecting.

According to an embodiment, each of the one or more ECG studies selected from a database of ECG studies comprise an analysis by an ECG analysis algorithm, and wherein both the first clinician and the second clinician receive the analysis by the ECG analysis algorithm during their review.

According to an embodiment, the first clinician and second clinician are presented with an automated analysis of each of the one or more ECG studies, performed by an ECG analysis algorithm.

According to an embodiment, the identified one or more differences between the first review and the second review are clinical differences.

According to an embodiment, the second clinician cannot access the first review, so that the second clinician can perform a naïve review. The first and second reviews can be assessed with the use of automated analysis of the differences and similarities between the reviews or with a third clinician.

According to an embodiment, the second clinician can access the first review in order to grade the first review explicitly.

According to an aspect is a system for reviewing ECG studies. The system includes: a database of ECG studies, comprising a plurality of ECG studies, wherein each of the plurality of ECG studies have been subjected to a first review by a first clinician, the first review comprising one or more first review elements, the one or more first review elements comprising one or more of an identity of the first clinician, a diagnosis, annotation of the ECG study, a note associated with the ECG study, and a measurement associated with the ECG study; a trained comparison algorithm configured to compare a first review and a second review for each of the one or more selected ECG studies; a processor configured to: (i) select one or more of the plurality of ECG studies from the database of ECG studies; (ii) present the one or more selected ECG studies to a second clinician for a second review, the second review comprising one or more second review elements, the one or more second review elements comprising one or more of an identity of the second clinician, a diagnosis, annotation of the ECG study, a note associated with the ECG study, and a measurement associated with the ECG study; (iii) compare, using the trained comparison algorithm, the first review and the second review for each of the one or more selected ECG studies to: (1) identify one or more differences between the first review and the second review, and/or (2) identify a degree of difference of each of the identified one or more differences between the first review and the second review; and (iv) generate a report summarizing the differences and/or degree of differences for the one or more selected ECG studies.

According to an embodiment, the processor is further configured to process the first review and second review to align the one or more first review elements and the one or more second review elements.

According to an embodiment, the processor is further configured to compare, using the trained comparison algorithm, the first review and the second review for each of the one or more selected ECG studies to rank the one or more selected ECG studies, wherein the rank is utilized to: (i) present the one or more selected ECG studies in a ranked order for comparison, and/or (ii) eliminate any of the one or more selected ECG studies that fall below a predetermined ranking.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a method for reviewing ECG studies, in accordance with an embodiment.

FIG. 2 is a schematic representation of an ECG review system, in accordance with an embodiment.

FIG. 3 is a flowchart of a method for training a comparison algorithm, in accordance with an embodiment.

FIG. 4 is a schematic representation of an ECG review system, in accordance with an embodiment.

### DETAILED DESCRIPTION OF EMBODIMENTS

The present disclosure describes various embodiments of a system and method configured for review of ECG studies using an ECG review system. More generally, Applicant has recognized and appreciated that it would be beneficial to provide methods and systems for more efficient and less burdensome quality control review of ECG analyses. Accordingly, an ECG review system selects one or more ECG studies from a database of ECG studies, wherein the one or more ECG studies have been subjected to a first review by a first clinician, and stores the selected ECG studies in a review database. The system then presents the one or more selected ECG studies to a second clinician for a second review, and stores the second review in the review database. A third entity compares the first review and the second review for each of the one or more selected ECG studies to: (i) identify one or more differences between the first review and the second review, and (ii) identify a degree of difference of each of the identified one or more differences between the first review and the second review. The system then generates a report summarizing the differences and degree of differences for the one or more selected ECG studies.

According to an embodiment, the methods and systems described or otherwise envisioned herein automate and improve the process of evaluating the diagnostic quality of a report on several levels, including reducing bias, improving the quality of the signal generated by the device, and giving guidance that helps physicians improve the accuracy of the diagnostic statements and measurements noted in the report. In addition, the methods and systems leverage the peer review aspect of the process to track, measure, and improve the quality of the algorithm itself. A system of audits and logs during adjudication is designed to expedite and improve the process of clearing the quality of new algorithms.

Thus, according to an embodiment, is a means of implementing a method to acquire a second read of already confirmed reports, in order to further educational opportunities for standardization of diagnoses. This second read is distinct from an overread, by which an attending physician reads and amends statements submitted by fellows and residents. This latter approach is a regular part of clinical workflow. A “second read” means a confirmed ECG will be pulled, cleaned of the read by the first physician, and then presented to a second physician for a second read. The results are stored in a training database, along with an initial ECG algorithmic read and the first physician's finalized read. Initially, the results are presented to a quality director (QD) for adjudication. Over time, this adjudication is a feedback signal where a cross-reader comparison metric can be improved for accuracy over time. This automated scoring mechanism has multiple uses, including but not limited to automatic ranking of semantically different diagnoses by severity to improve QD workflow, improving the algorithmically generated preliminary reads (i.e., algorithm-generated diagnostic statements), and can act as a feedback signal for an algorithm that can parse statements based on clinical semantic meaning and not simple textual or syntactic differences.

According to an embodiment, the methods and systems herein comprise a general-purpose tool to capture, in a systematic way, deviations between physicians from algorithmic analysis and between physicians themselves. This builds on the diagnostic workflow capability of existing systems by adding a reporting quality module. The module allows for a quality director to set parameters for pulling sets of ECGs, at recurring frequency, and assigning to a quality review inbox. Any ECGs in this review inbox is then read as normal; all reads are stored in a way so that the algorithm, reader 1, and each subsequent readers' decisions can be analyzed post-hoc. Having each statement set also allows for further analyses to derive a diagnostic difference/significance score, further ranking the types of differences. Finally, the readings and scores can be presented to the quality director, who can adjudicate the differences. The module will also enable summary analytics, facilitate training opportunities, while also improving diagnostic standardization. With the differences and adjudication, existing ECG systems can also benefit from the annotation, which can be fed back for algorithm improvements. This framework can be extended to other modalities and products. The framework is the same for both on-premise and cloud deployments. Once in the cloud, additional de-identification and pooling can be made available to categorize ECGs for further annotation. Finally, this same UI can be provided to university hospitals, medical schools, and practitioners who can sign on to grade/read ECGs. This enables further algorithm development and better measurements of diagnostic variance.

According to an embodiment, the systems and methods described or otherwise envisioned herein can, in some non-limiting embodiments, be implemented as a component of or an extension of an ECG system such as the Philips® IntelliSpace® system (available from Koninklijke Philips NV, the Netherlands), or as an element for a commercial product for ECG analysis or review, or any suitable system. However, the disclosure is not limited to these devices or systems, and thus disclosure and embodiments disclosed herein can encompass any system that may utilize or benefit from quality control analysis or review, including but not limited different modalities such as imaging using ultrasound, X-ray, MRI, CT/PET, or visible light and fluorescence microscopy, or blood tests, genetic tests, and so forth.

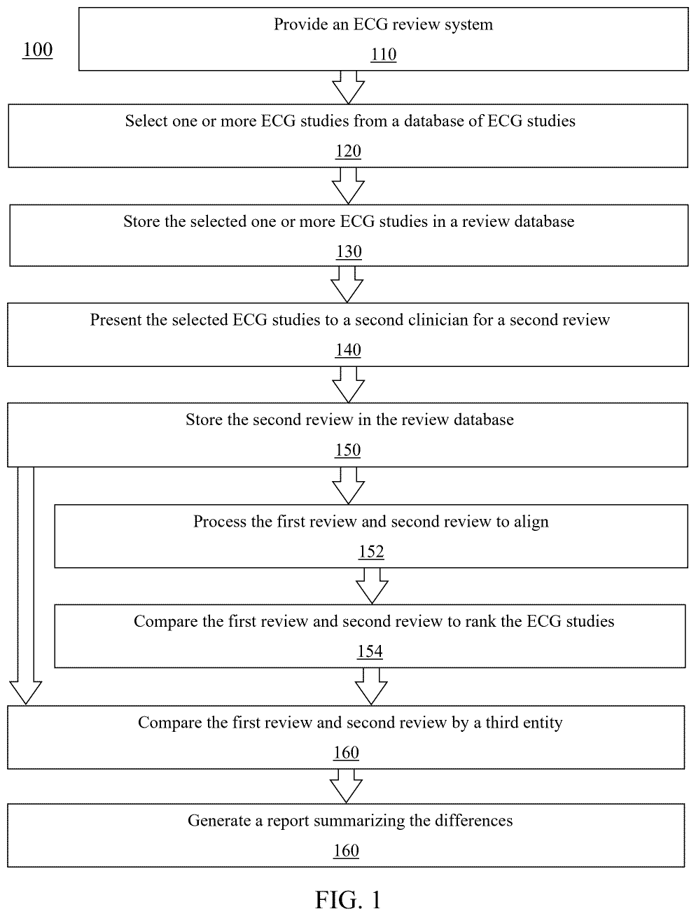

Referring to FIG. 1, in one embodiment, is a flowchart of a method 100 for reviewing ECG studies using an ECG review system. The methods described in connection with the figures are provided as examples only, and shall be understood to not limit the scope of the disclosure. The ECG review system can be any of the systems described or otherwise envisioned herein. The ECG review system can be a single system or multiple different systems.

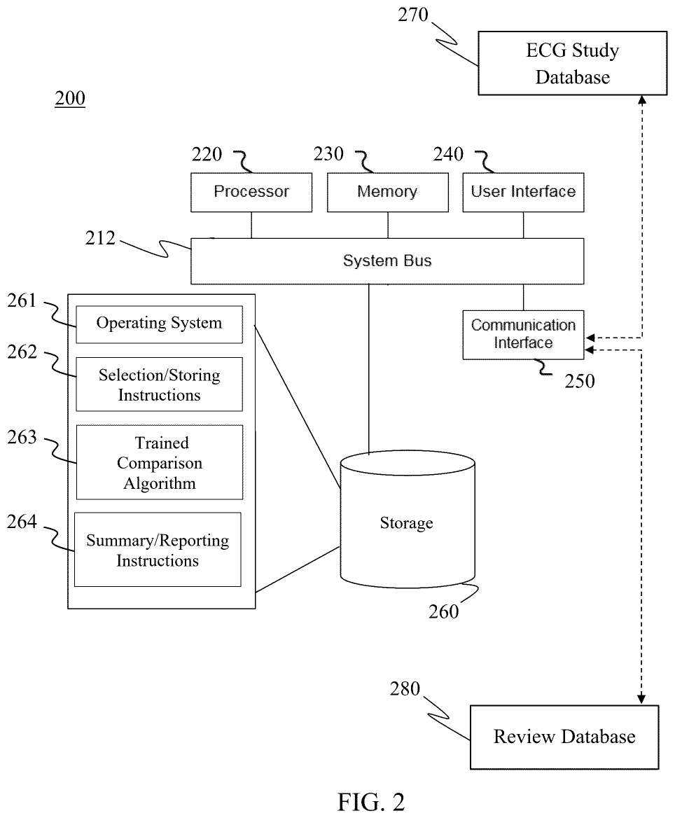

At step 110 of the method, an ECG review system 200 is provided. Referring to an embodiment of an ECG review system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, ECG review system 200 can be any of the systems described or otherwise envisioned herein. Other elements and components of ECG review system 200 are disclosed and/or envisioned elsewhere herein.

According to an embodiment, the ECG review system 200 is or comprises or is in local and/or remote communication with a database 270 of ECG studies as described or otherwise envisioned herein. According to an embodiment, database 270 of ECG studies comprise a plurality of ECG studies that have been subjected to a first review by a first clinician.

According to an embodiment, the ECG review system 200 is or comprises or is in local and/or remote communication with a review database 280 as described or otherwise envisioned herein. According to an embodiment, review database 280 comprises a plurality of ECG studies selected from the plurality of ECG studies in the database 270 of ECG studies. The selected plurality of ECG studies in the review database comprise information about the first review by the first clinician, and will ultimately comprise information about a second review by a second clinician, as described or otherwise envisioned herein.

At step 120 of the method, the ECG analysis system selects one or more ECG studies from the database 270 of ECG studies. Each of these ECG studies comprises an ECG trace obtained from a respective subject. These selected ECG studies are selected by the ECG analysis system for review. The review may be for any of a plurality of different purposes, as described or otherwise envisioned herein. For example, the review may be for quality control, clinician review and/or assessment, care setting review and/or assessment, clinician training, algorithm training, and any of a wide variety of other purposes.

According to an embodiment, the selected one or more ECG studies from the database 270 of ECG studies have been subjected to a first review by a first clinician. The first review by the first clinician includes one or more first review elements, which is information added to the ECG trace. The one or more first review elements can comprise many different elements and information. According to an embodiment, the one or more first review elements comprise one or more of an identity of the first clinician, a diagnosis, annotation of the ECG study, a note associated with the ECG study, and a measurement associated with the ECG study. Since review elements are always stored in the quality system, any combinations of review elements are possible. According to an embodiment, no review elements need be shown to the reviewer to force a de novo diagnosis. According to an embodiment, only the first diagnosis is shown to the reviewer so that the reviewer can grade and score the original diagnosis.

Selection of ECG studies from the database by the ECG analysis system can be done in a variety of different ways. According to an embodiment, the ECG analysis system randomly (or semi-randomly) selects the ECG studies. This can be done using any process for randomly selecting items from a data structure. For example, the ECG analysis system can be programmed or otherwise designed to randomly select a predetermined number of ECG studies from the ECG database. The selection can be done periodically in accordance with a predetermined calendar or schedule, or it can be done in response to a command or a query to perform the selection. Other options are possible.

According to an embodiment, the selection of the ECG studies is partially or wholly constrained by predetermined or selected/identified selection criteria. These selection criteria can be provided, for example, by a user of the ECG analysis system, such as via a user interface. The selection criteria could be any criteria that distinguishes between two or more ECG studies. Examples of selection criteria include age and/or gender of the subject, diagnostic statements, specific measurements, and many other criteria.

According to an embodiment, the selected one or more ECG studies from the database 270 of ECG studies have been previously analyzed by a computer-based ECG interpretation algorithm. There are many computer-based ECG interpretation algorithms known in the art. Generally, these algorithms perform an initial analysis or review of an ECG trace and generate information about that ECG trace or subject. This information can thus be provided to clinicians—such as the first clinician and second clinician described herein—as part of their analysis (and thus as part of the review process as described or otherwise envisioned herein).

At step 140 of the method, the ECG analysis system stores the selected one or more ECG studies in a review database 280. The ECG studies can be stored in the review database using any method for storing data. The selected one or more ECG studies are stored in the review database with the first review, or are stored in a way that links or associates the stored ECG studies with the first reviews for those ECG studies.

According to an embodiment, the review database 280 is a dedicated quality database or data structure, which can be called for example a “QualityDB.” According to an embodiment, since these ECGs have already been read and billed, they are stored separately from other ECGs so that a patient is not erroneously re-billed. Because this quality review system is a separate module of the diagnostic system, clinicians can also focus on providing healthcare by reading new ECG reads before providing the second review to the quality module as discussed below.

At step 140 of the method, the ECG analysis system presents one or more of the selected one or more ECG studies to a second clinician for a second review. Thus, according to an embodiment, the ECG analysis system may comprise, or be in local and/or remote communication with, a user interface that allows for review of an ECG study. According to an embodiment, the second clinician performs the second review according to methods known in the art for reviewing an ECG trace or study. The second review by the first clinician includes one or more second review elements, which is information added to the ECG trace. The one or more second review elements can comprise many different elements and information. According to an embodiment, the one or more second review elements comprise one or more of an identity of the second clinician, a diagnosis, annotation of the ECG study, a note associated with the ECG study, and a measurement associated with the ECG study. Since review elements are stored in the quality system, many combinations of review elements are possible. According to an embodiment, no review elements need be shown to the reviewer to force a de novo diagnosis. According to an embodiment, the first diagnosis is shown to the reviewer so that the reviewer can grade and score the original diagnosis.

According to an embodiment, the second clinician cannot access the first review before or during performance of the second review (or optionally at any point). Thus, in accordance with this embodiment, when the ECG analysis system presents the ECG studies to a second clinician for a second review, it provides only the ECG trace for that ECG study, and/or other information such as the result of the analysis by the computer-based ECG interpretation algorithm. However, the ECG analysis system either strips out some or all of the first review, or otherwise obscures the information or prevents the second clinician from seeing, reviewing, or otherwise getting access to the first review.

According to another embodiment, the second clinician can access the first review before or during performance of the second review (or optionally at any point). Thus, during the second review, the second clinician may access or review one or more first review elements from the first review. Thus, the second clinician may identify similarities and/or differences between the first review and the second review as part of the second review. Thus, the second clinician can grade and score the first review without making a second diagnosis. Many other options are possible.

At step 150 of the method, the ECG analysis system stores the second review of the selected one or more ECG studies in the review database 280. The second review can be stored in the review database using any method for storing data. The second reviews are stored in the review database together with the corresponding first review and ECG study, or are stored in a way that links or associates the second reviews with the corresponding first review and ECG study.

According to an embodiment, at optional step 152 of the method, the ECG analysis system processes the first review and second review to align the one or more first review elements and the one or more second review elements. For example, in some platforms, some or all review elements may be associated with a diagnostic finding code or other standardized component. The ECG analysis system can utilizes these standardized components to align or reorder the one or more first review elements and the one or more second review elements, such that subsequent review of the first review and second review will be faster and more efficient.

According to an embodiment, at optional step 154 of the method, the ECG analysis system compares the first review and the second review for some or all of the one or more selected ECG studies to rank these one or more selected ECG studies relative to one another. For example, the system can utilize second reviews of interest (i.e. those reviews with the biggest consequential diagnostic differences, etc.) to rank the one or more selected ECG studies relative to one another, and/or to eliminate ECG studies if the difference between the first review and the second review are not significant or fall below a difference threshold. If the ECG studies are ranked or eliminated, this can make subsequent review of the first review and second review faster and more efficient. Thus, according to an embodiment, the ECG analysis system can present the one or more selected ECG studies in a ranked order for comparison, and/or can eliminate any of the one or more selected ECG studies that fall below a predetermined ranking.

According to an embodiment, the ECG analysis system compares the first review and the second review for some or all of the one or more selected ECG studies utilizing a trained comparison algorithm. This comparison algorithm can be trained, for example, to compare reviews and rank or eliminate ECG studies as described or otherwise envisioned herein.

At step 160 of the method, a third entity different from the first clinician and second clinician receives one or more of the twice-reviewed ECG studies stored in the review database 280, for an analysis. According to an embodiment, the third entity compares the first review and the second review for the received one or more selected ECG studies. The comparison can be performed many ways, as described or otherwise envisioned herein. The comparison can be designed, configured, or intended to identify similarities (where “similarities” can be similar or identical to each other) and/or differences between the two reviews. The comparison can be a comparison of one or more of the review elements of the first review to one or more of the review elements of the second review.

According to an embodiment, the review by the third entity is intended to identify one or more differences between the first review and the second review. According to an embodiment, the review is intended to identify a degree of difference of each of the identified one or more differences between the first review and the second review. Other outcomes of the review by the third entity are possible.

According to an embodiment, the third entity is a third clinician, different from the first clinician and the second clinician. The third clinician could utilize, for example, an adjudication viewer which enables comparison of the first review and second review. The adjudication viewer, which is a user interface, can be programmed or designed to allow the third clinician to compare the reviews in a wide variety of different ways.

According to an embodiment, the third entity is a trained comparison algorithm. The comparison algorithm can be trained, for example, to compare the first review and the second review to identify one or more differences between the first review and the second review, and/or to identify a degree of difference of each of the identified one or more differences between the first review and the second review, among other possible outcomes.

The trained comparison algorithm can be any model that can be trained to utilize the input to generate the output, as described or otherwise envisioned herein. For example, the trained comparison algorithm can be a neural network or other trained machine learning model. Thus, according to an embodiment, the ECG analysis system comprises a trained comparison algorithm that receives the input data (e.g., the first review and second review) and outputs data regarding a comparison of the reviews.

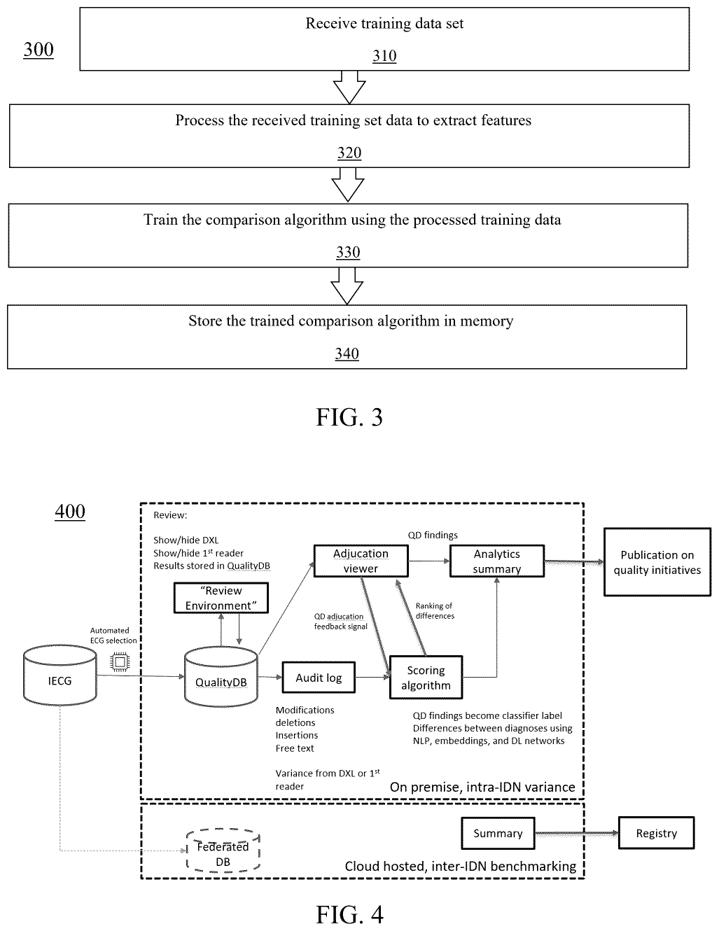

The trained comparison algorithm can be trained in a variety of different ways. According to one embodiment, the comparison algorithm is trained in an unsupervised manner or in a supervised manner. Referring to FIG. 3, in one embodiment, is a flowchart of a method 300 for training the comparison algorithm of the ECG analysis system 200. This method may be performed by the ECG analysis system or may be performed by another system such as a specialized machine learning model training system.

At step 310 of the method, the training system receives training data which will be used to train the model. The training data can be any data sufficient to train the model to utilize the described input data to generate the described output. For example, the training data may comprise first and second reviews for a plurality of ECG studies for a plurality of patients, including with ground truth optimization. This training data, which could be utilized in a supervised or unsupervised manner, can comprise 100s or 1000s of ECG studies, and can optionally be updated with new studies. The training data may also comprise other information. This training data may be obtained and curated by an expert such as a clinician, or it may be obtained and curated under the supervision of a clinician, or it may be obtained and utilized without curation. The training data may be received from any source. For example, the training data may be received from an electronic medical record database or system, or any other component of the ECG analysis system or a training system. According to an embodiment, the ECG analysis system 200 comprises or is in direct or indirect communication with a database which comprises some or all of the training data set.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 320 of the method, the training system trains the comparison algorithm, using the training data, to identify similarities and/or differences between first reviews and second reviews for a plurality of ECG studies, and to generate a degree of difference between information in first reviews and second reviews for a plurality of ECG studies. The comparison algorithm is trained using any method for training such a model. The trained comparison algorithm is a unique model based on the training data used to train the model. Following training, the system comprises a trained comparison algorithm.

Thus, following training, the comparison algorithm is a specialized model configured to receive the input (namely, first reviews and second reviews) and generate the very specific output, namely the similarities and/or differences between first reviews and second reviews, and a degree of difference between information in first reviews and second reviews.

At step 330 of the method, the trained comparison algorithm is stored for future use. According to an embodiment, the trained comparison algorithm may be stored in local or remote storage.

Returning to method 100 in FIG. 1, at step 170 of the method, the system generates a summary or report of the outcome(s) of step 160 of the method in which a third entity different from the first clinician and second clinician compares the first review and the second review for the received one or more selected ECG studies to identify similarities (where “similarities” can be similar or identical to each other) and/or differences between the two reviews. This comparison can be a comparison of one or more of the review elements of the first review to one or more of the review elements of the second review. Accordingly, the report generated by the system can comprise the differences and/or degree of differences for the one or more selected ECG studies, and/or a summary thereof. The report can further comprise any of the other information provided to the ECG analysis system or generated by the ECG analysis system. For example, the report may comprise information about a subject or subjects, review elements from any of the three reviews, and/or any other information.

The report generated in step 170 of the method can be utilized immediately, or it can be stored in local or remote storage for future use. According to an embodiment, generated report may be provided to a user via any mechanism for providing information. According to an embodiment, the report may be communicated by wired and/or wireless communication to a user interface and/or to another device. For example, the system may communicate the report to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. Other reporting methods are possible.

### EXAMPLE

The following is provided as an example of the methods and systems described or otherwise envisioned herein. Thus, it will be understood that this example is a non-limiting example.

Referring to FIG. 4, in one embodiment, is a schematic representation of a system and method for reviewing ECG studies using an ECG analysis system. According to an embodiment, the ECG analysis system comprises a randomizer or randomizing module that copies a set of confirmed ECG studies into a dedicated quality database (termed, for example, the QualDB). Elements of the previous review (including but not limited to notes, interpretations, measurements, demographics, the first reader's identity, etc.) are stored in the QualDB. Notably, these elements are hidden. The second reviewer will only see review elements such as the ECG, age, gender, and the outputs of the DXL algorithm, similar to the first reviewer. According to an embodiment, the randomizer has a configuration table where the quality director (QD) can set filters to narrow the range of studies for QualDB (filters can be elements such as age, gender, diagnostic statements, specific measurements, etc.). According to an embodiment, the QD can set number of ECGs and frequency (for example, 50 ECGs will be sent to quality module, once per week, although many other numbers and frequencies are possible).

According to an embodiment, the selected ECGs stored in the QualDB will be placed in a dedicated peer review space of a Review Environment. Separating these ECGs can be crucial. Since these ECGs had already been read and billed, they must remain separate so that the patient is not erroneously re-billed. They physician can also focus on the new and/or urgent ECG reads before they provide dedicated second reads. According to an embodiment, the second reviewers review is also stored within the QualDB.

Accordingly, the ECG analysis system creates a repository of first and second reviews (stored as audit logs). The system has the reviewer's identities and the precise modifications to algorithmic reviews, and thus can how the reviewers may (or may not) differ. This includes the data elements that allow for comparisons between reviewers, comparing reviewers to the algorithmic reviews, and tracking whether the differences have meaningful clinical significance.

According to an embodiment, there are at least two approaches in enabling analysis of these diagnostic variance. The first is to present the first and second reads to the QD within the Adjudication Viewer. In the review module, the QD can see the ECG, statements, and clinician modifications and measurements, side-by-side, from both reviewers. In some platforms, each interpretation corresponds to a diagnostic finding code. Using these codes will allow for removing trivial differences based on differences in order of statements. Reordering the statements will help speed up the QD's adjudication. The QD thus adjudicates if the reviews agree, agree somewhat, or disagree, for example. The QD can further adjudicate the reviews to their own experience, and thus it becomes a third review. During this review phase, the system can provide two scoring boxes including one for the correct diagnoses (both, first reviewer, second reviewer, or neither), and one for the degree of difference.

According to an embodiment, a second approach is to calculate a quality score, such as through the use of a quality score algorithm. The quality score may not simply be a textual or syntactic difference; it can reflect an actual semantic difference with respect to patient care. However, simply providing a syntactic difference score can be an important first step. As in the QD scoring method, the system can remove the trivial order differences (e.g., using the finding code match). According to an embodiment, one can create a library of findings that are equivalent (e.g., this can be done with internal clinical specialists). Using this equivalence mapping, the system can subtract out equivalent diagnostic statements. The system can also subtract out literal matches. The remaining diagnoses will be enriched in potential clinical differences. To generate a final score, the system can then do a textual similarity match (in this case, a syntactic match). Since the system has first subtracted out the equivalent statements, the remaining differences can be scored in this way. The fewer matches, the greater the likelihood of diagnostic differences.

According to a second approach (e.g., an algorithmic quality scoring approach) the system can further pre-rank differences. In this way, the system can automate the ranking of second reviews of interest (e.g., those reads with the greatest consequential diagnostic differences). This can speed up subsequent review by the QD.

Thus, whether the quality score is manually (by the QD) or algorithmically derived, the system can convert the individual quality score into a performance metric, which can then be utilized for many purposes including for baselining and tracking care standardization efforts in an analytics summary. As a note, the Audit Log, Adjucation Viewer, Scoring Algorithm, and Analytics Summary are all modules (e.g., containing data tables and compute processes) within the QualDB and/or a processor of the system.

According to an embodiment, the review module can be placed in the cloud. Specifically, rather than ECGs being copied to an on-premise QualityDB server, the server can reside in an online data warehouse. Keeping the methods and systems on-premise may be a good approach for a hospital (or integrated delivery network-IDN) to track their own quality initiatives. The cloud-based embodiment enables a federated approach, where multiple hospitals and IDNs can place a subset of ECGs into the cloud. The ECGs will be de-identified, with only the ECG and initial algorithm review information provided (the same as for the on-premise system). However, in this approach, physicians from multiple hospitals can read the same ECG, providing inter-hospital comparisons. With the proper adjustments to performance metrics, this can serve as the basis for registry-driven standardization of care and benchmarking applications (e.g., day-to-day quality management).

According to an embodiment, the diagnostic statement equivalence library for the quality scoring module can be created by supervised training NLP and deep-learning methods. The Quality Director (QD) scores can be used as annotations. By creating embeddings from the set of diagnostic statements that are not significantly clinical different, these pairs of embeddings become a match. One deep-learning approach is to use the autoencoder method from a transformer architecture to compare embeddings.

According to an embodiment, the QD can use scoring results to train an ML or DL model to translate the ECG into the findings. The annotations by the QD can be as follows: the model returns a match if any of the findings are returned from the ECG, where the QD score was agree or somewhat agree. The model can return multiple results since ECGs can contain multiple findings. This implies that, for a given ECG, those codes are correct or has a difference with no clinical significance. This quality module can be added to the source diagnostic platform, where the quality module can compare the first reader's initial read to the stored QualDB. Having this quality module to generate findings from ECGs based on the scoring feedback allows for the module to act as guardrail during an initial read. A physician who deviates from the canonical read can see a suggestion popup, with the QD's recommendation.

According to an embodiment, an ML/DL model can be trained on the ECGs themselves, especially those proven to exhibit diagnostic variance with severe clinical consequence. In this case, the training annotation is a single classifier for those ECGs that proved problematic. This trained model can act as a guardrail during an initial review, where ECGs matched under this model can be recommended automatically for an over-review within a diagnostic workflow. The QD's recommendation can also pop-up appear to provide additional guidance. Finally, these ECGs can be automatically sent into system for additional training and analysis.

According to an embodiment, an NLP/DL approach for comparing findings enables enable automated calculation of a quality score. Rather than a finding-by-finding comparison, the similarity of the embeddings generated from the findings can be calculated by a distance metric (e.g., the closer the distance, the more similar). This can facilitate pre-ranking and presenting a quality review worklist so that the QD sees the largest differences first.

According to an embodiment, the equivalence library is not a match of findings-to-findings, but findings to a reference language (e.g. UMLS with SNOMED). By decomposing finding codes into structure-location-observation-state hierarchy, equivalence comparisons can be facilitated. One initial attempt to build the library is to use findings that are syntactically dissimilar but semantically similar. This set of codes can be the object of focus in translating into the reference language. Such a library becomes training annotations for NLP/DL based modelling, for future automation. The key point of this embodiment is that the findings are translated into a reference language.

Referring to FIG. 2 is a schematic representation of an ECG analysis system 200. System 200 may be any of the systems described or otherwise envisioned herein, and may comprise any of the components described or otherwise envisioned herein. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, the system may comprise, among other instructions or data, selection and/or storing instructions 262, a trained comparison algorithm 263, and/or summary/reporting instructions 264.

According to an embodiment, selection and/or storing instructions 262 direct the system to select one or more ECG studies from the database 270 of ECG studies. Each of these ECG studies comprises an ECG trace obtained from a respective subject. These selected ECG studies are selected by the ECG analysis system for review. Selection of ECG studies from the database by the ECG analysis system can be done in a variety of different ways. According to an embodiment, the ECG analysis system randomly (or semi-randomly) selects the ECG studies. This can be done using any process for randomly selecting items from a data structure. For example, the ECG analysis system can be programmed or otherwise designed to randomly select a predetermined number of ECG studies from the ECG database. The selection can be done periodically in accordance with a predetermined calendar or schedule, or it can be done in response to a command or a query to perform the selection. Other options are possible. According to an embodiment, the selection of the ECG studies is partially or wholly constrained by predetermined or selected/identified selection criteria. These selection criteria can be provided, for example, by a user of the ECG analysis system, such as via a user interface. The selection criteria could be any criteria that distinguishes between two or more ECG studies. Examples of selection criteria include age and/or gender of the subject, diagnostic statements, specific measurements, and many other criteria.

According to an embodiment, the trained comparison algorithm 263 is a trained model utilized as a third entity to compare the first review and the second review to identify one or more differences between the first review and the second review, and/or to identify a degree of difference of each of the identified one or more differences between the first review and the second review, among other possible outcomes. The trained comparison algorithm can be any model that can be trained to utilize the input to generate the output, as described or otherwise envisioned herein. For example, the trained comparison algorithm can be a neural network or other trained machine learning model. Thus, according to an embodiment, the ECG analysis system comprises a trained comparison algorithm that receives the input data (e.g., the first review and second review) and outputs data regarding a comparison of the reviews.

According to an embodiment, trained comparison algorithm 263 is trained according to the methods and systems described or otherwise envisioned herein to analyze first and second reviews and generate a comparison. Thus, the trained comparison algorithm and the ECG analysis system is configured to process many thousands or millions of datapoints in the input data used to train the comparison algorithm, as well as to process and analyze first and second reviews for a plurality of ECG studies (in order to generate the resulting comparison as output). For example, generating a functional and skilled trained algorithm using an automated process such as feature identification and extraction and subsequent training requires processing of millions of datapoints from input data and the generated features. This can require millions or billions of calculations to generate a novel trained comparison algorithm from those millions of datapoints and millions or billions of calculations. As a result, each trained algorithm is novel and distinct based on the input data and parameters of the machine learning algorithm, and thus improves the functioning of the ECG analysis system. Thus, generating a functional and skilled trained classifier comprises a process with a volume of calculation and analysis that a human brain cannot accomplish in a lifetime, or multiple lifetimes.

According to an embodiment, summary/reporting instructions 264 direct the system to generate a summary or report of the outcome(s) of the method in which a third entity different from the first clinician and second clinician compares the first review and the second review for the received one or more selected ECG studies to identify similarities (where “similarities” can be similar or identical to each other) and/or differences between the two reviews. This comparison can be a comparison of one or more of the review elements of the first review to one or more of the review elements of the second review. Accordingly, the report generated by the system can comprise the differences and/or degree of differences for the one or more selected ECG studies, and/or a summary thereof. The report can further comprise any of the other information provided to the ECG analysis system or generated by the ECG analysis system. For example, the report may comprise information about a subject or subjects, review elements from any of the three reviews, and/or any other information.

According to an embodiment, summary/reporting instructions 264 direct the system to provide the generated report or summary to a user. According to an embodiment, generated report may be provided to a user via any mechanism for providing information. According to an embodiment, the report may be communicated by wired and/or wireless communication to a user interface and/or to another device. For example, the system may communicate the report to a mobile phone, computer, laptop, wearable device, and/or any other device configured to allow display and/or other communication of the report. The user interface can be any device or system that allows information to be conveyed and/or received, and may include a display, a mouse, and/or a keyboard for receiving user commands. Other reporting methods are possible.

The present invention may be a system, a method, and/or a computer program product. The computer program product may include a non-transitory computer readable storage medium (or media) having computer readable program instructions thereon for causing a system or processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any combination of the foregoing, among other possibilities. Computer readable program instructions described herein can be downloaded to respective computing/processing devices from a computer readable storage medium or to an external computer or external storage device via a network, for example, the internet, a local area network, and/or a wireless network. Aspects of the present invention are described herein with reference to flowchart illustrations and/or block diagrams of methods, apparatus, systems, and computer program products according to embodiments of the invention. It will be understood that each block of the flowchart illustrations and/or block diagrams, and combinations of blocks in the flowchart illustrations and/or block diagrams, can be implemented by computer readable program instructions.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for reviewing ECG studies, comprising:
selecting one or more ECG studies from a database of ECG studies, wherein the one or more ECG studies have been subjected to a first review by a first clinician, the first review comprising one or more first review elements, the one or more first review elements comprising one or more of an identity of the first clinician, a diagnosis, annotation of the ECG study, a note associated with the ECG study, and a measurement associated with the ECG study;
storing the selected one or more ECG studies, including the first review, in a review database;
presenting the one or more selected ECG studies to a second clinician for a second review, the second review comprising one or more second review elements, the one or more second review elements comprising one or more of an identity of the second clinician, a diagnosis, annotation of the ECG study, a note associated with the ECG study, and a measurement associated with the ECG study;
storing the second review in the review database;
comparing, by a third entity different from the first clinician and second clinician, the first review and the second review for each of the one or more selected ECG studies to: (i) identify one or more differences between the first review and the second review, and/or (ii) identify a degree of difference of each of the identified one or more differences between the first review and the second review;
generating a report summarizing the differences and/or degree of differences for the one or more selected ECG studies.

2. The method of claim 1, further comprising processing the first review and second review to align the one or more first review elements and the one or more second review elements.

3. The method of claim 1, further comprising comparing, by a trained comparison algorithm, the first review and the second review for each of the one or more selected ECG studies to rank the one or more selected ECG studies, wherein the rank is utilized to: (i) present the one or more selected ECG studies in a ranked order for comparison, and/or (ii) eliminate any of the one or more selected ECG studies that fall below a predetermined ranking.

4. The method of claim 1, wherein the third entity is a third clinician, and the comparing is performed manually by the third clinician using an adjudication viewer.

5. The method of claim 1, wherein the third entity is a trained comparison algorithm.

6. The method of claim 1, wherein the report comprises a performance metric based on the summarized differences and degree of differences for the one or more selected ECG studies.

7. The method of claim 1, wherein the one or more ECG studies are randomly selected from the database of ECG studies.

8. The method of claim 1, wherein the one or more ECG studies are selected from the database of ECG studies based on one or more criteria, the one or more criteria determined via a user interface prior to selecting.

9. The method of claim 1, wherein each of the one or more ECG studies selected from a database of ECG studies comprise an analysis by an ECG analysis algorithm, and wherein both the first clinician and the second clinician receive the analysis by the ECG analysis algorithm during their review.

10. The method of claim 1, wherein the first clinician and second clinician are presented with an automated analysis of each of the one or more ECG studies, performed by an ECG analysis algorithm.

11. The method of claim 1, wherein the identified one or more differences between the first review and the second review are clinical differences.

12. The method of claim 1, wherein the second clinician cannot access the first review.

13. A system reviewing ECG studies, comprising:
a database of ECG studies, comprising a plurality of ECG studies, wherein each of the plurality of ECG studies have been subjected to a first review by a first clinician, the first review comprising one or more first review elements, the one or more first review elements comprising one or more of an identity of the first clinician, a diagnosis, annotation of the ECG study, a note associated with the ECG study, and a measurement associated with the ECG study;
a trained comparison algorithm configured to compare a first review and a second review for each of the one or more selected ECG studies;
a processor configured to: (i) select one or more of the plurality of ECG studies from the database of ECG studies; (ii) present the one or more selected ECG studies to a second clinician for a second review, the second review comprising one or more second review elements, the one or more second review elements comprising one or more of an identity of the second clinician, a diagnosis, annotation of the ECG study, a note associated with the ECG study, and a measurement associated with the ECG study; (iii) compare, using the trained comparison algorithm, the first review and the second review for each of the one or more selected ECG studies to: (1) identify one or more differences between the first review and the second review, and/or (2) identify a degree of difference of each of the identified one or more differences between the first review and the second review; and (iv) generate a report summarizing the differences and/or degree of differences for the one or more selected ECG studies.

14. The system of claim 13, wherein the processor is further configured to process the first review and second review to align the one or more first review elements and the one or more second review elements.

15. The system of claim 13, wherein the processor is further configured to compare, using the trained comparison algorithm, the first review and the second review for each of the one or more selected ECG studies to rank the one or more selected ECG studies, wherein the rank is utilized to: (i) present the one or more selected ECG studies in a ranked order for comparison, and/or (ii) eliminate any of the one or more selected ECG studies that fall below a predetermined ranking.
